(19) United States
(12) Patent Application Publication
(10) Pub. No.: US 2024/0245350 A1
(43) Pub. Date: Jul. 25, 2024

(54) VITAL SIGNS INFORMATION PROCESSING APPARATUS, VITAL SIGNS INFORMATION PROCESSING METHOD, AND NON-TRANSITORY COMPUTER READABLE STORAGE MEDIUM

(71) Applicant: NIHON KOHDEN CORPORATION, Tokyo (JP)

(72) Inventors: Hideki OCHIAI, Tokorozawa-shi (JP); Minoru MATSUSHIMA, Tokorozawa-shi (JP); Tetsuri ARIYAMA, Tokorozawa-shi (JP)

(73) Assignee: NIHON KOHDEN CORPORATION, Tokyo (JP)

(21) Appl. No.: 18/415,237

(22) Filed: Jan. 17, 2024

(30) Foreign Application Priority Data
Jan. 25, 2023 (JP) 2023-009556

(51) Int. Cl. A61B 5/00; A61B 5/026; A61B 5/352; G16H 50/30

(52) U.S. Cl. CPC A61B 5/4824; A61B 5/026; A61B 5/352; G16H 50/30; A61B 2503/045

(57) ABSTRACT

A vital signs information processing apparatus includes an obtaining unit configured to obtain vascular dynamic information indicating a circulation state of blood of a subject; and a processor configured to perform a normalization processing on the vascular dynamic information to obtain an index value of pain evaluation of the subject. The processor is configured to calculate a reference value, based on the vascular dynamic information obtained in a reference period before a start of a treatment event related to a treatment for the subject, calculate a target value, based on the vascular dynamic information obtained after the start of the treatment event, and calculate a ratio of the target value to the reference value as the index value, as the normalization processing.

### CROSS-REFERENCE TO RELATED APPLICATIONS

This application is based on and claims priority under 35 USC 119 from Japanese Patent Application No. 2023-009556 filed on Jan. 25, 2023, the entire content of which is incorporated herein by reference.

### TECHNICAL FIELD

The presently disclosed subject matter relates to a vital signs information processing apparatus, a vital signs information processing method, and a non-transitory computer readable storage medium storing a vital signs information processing program.

### BACKGROUND ART

Most preterm infants and premature infants (hereinafter referred to as preterm infants and the like) receive treatments and examinations in neonatal intensive care units (NICUs), and the treatments and the examinations for preterm infants may require painful procedures. It is said that the experience of pain during the neonatal period may have a negative effect on sensitivity to pain and to the stress system of the autonomic nervous system. On the other hand, newborns cannot communicate well, and thus they cannot express their pain. Thus, there is a need for means by which medical workers can accurately understand the pain of newborns during painful treatment events.

In this regard, Non-Patent Literature 1 (Mio Ozawa, Naoko Sunagane, Katsuya Suda, Michio Hirata, Isao Kusakawa, Chieko Suzuki, “Verification of Practical Use of Japanese Version of Premature Infant Pain Profile”, Child Health Research Vol. 71, No. 1, 2012, p. 10-16) discloses a verification result regarding the premature infant pain profile (PIPP), which uses a total of seven indicators including two background indicators, two physiological indicators, and three facial expression indicators.

When using the PIPP or the premature infant pain profile revised (PIPP-R), which is an improved version of the PIPP, for preterm infants and the like, three facial expression indicators including “raised eyebrows” “closed eyes” and “nasolabial folds” need observation and evaluation for 30 seconds. For this reason, the burden on an evaluator who performs the evaluation is heavy. Furthermore, the evaluation of facial expressions tends to vary depending on evaluators.

### SUMMARY OF INVENTION

Aspect of non-limiting embodiments of the present disclosure relates to provide a vital signs information processing apparatus, a vital signs information processing method, and a non-transitory computer readable storage medium storing a vital signs information processing program that can reduce load on an evaluator who performs pain evaluation and reduce a variation in evaluation depending on evaluators.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to a first aspect of the present disclosure, there is provided a vital signs information processing apparatus including:
an obtaining unit configured to obtain vascular dynamic information indicating a circulation state of blood of a subject; and
a processor configured to perform a normalization processing on the vascular dynamic information to obtain an index value of pain evaluation of the subject,
in which the processor is configured to:
   calculate a reference value, based on the vascular dynamic information obtained in a reference period before a start of a treatment event related to a treatment for the subject;
   calculate a target value, based on the vascular dynamic information obtained after the start of the treatment event; and
   calculate a ratio of the target value to the reference value as the index value, as the normalization processing.

According to a second aspect of the present disclosure, there is provided a vital signs information processing method of a vital signs information processing apparatus, the vital signs information processing method including:
obtaining vascular dynamic information indicating a circulation state of blood of a subject; and
performing a normalization processing on the vascular dynamic information to obtain an index value of pain evaluation of the subject,
in which in the performing:
   a reference value is calculated based on the vascular dynamic information obtained in a reference period before a start of a treatment event related to a treatment for the subject;
   a target value is calculated based on the vascular dynamic information obtained after the start of the treatment event; and
   a ratio of the target value to the reference value is calculated as the index value, as the normalization processing.

According to a third aspect of the present disclosure, there is provided a non-transitory computer readable storage medium storing a vital signs information processing program used in a vital signs information processing apparatus, the program including instructions, when executed by a computer, causing the computer in the vital signs information processing apparatus to:
obtaining vascular dynamic information indicating a circulation state of blood of a subject; and
perform a normalization processing on the vascular dynamic information to obtain an index value of pain evaluation of the subject,
in which in the performing:
   a reference value is calculated based on the vascular dynamic information obtained in a reference period before a start of a treatment event related to a treatment for the subject;
   a target value is calculated based on the vascular dynamic information obtained after the start of the treatment event; and
   a ratio of the target value to the reference value is calculated as the index value, as the normalization processing.

### BRIEF DESCRIPTION OF DRAWINGS

Exemplary embodiment(s) of the present invention will be described in detail based on the following figures, wherein:

FIG. 1 illustrates an example of use of a processing apparatus according to an embodiment;

FIG. 2 illustrates a configuration of the processing apparatus illustrated in FIG. 1;

FIG. 3 is a graph illustrating an ECG waveform indicated by ECG data output from an obtaining unit illustrated in FIG. 2;

FIG. 4 is a graph illustrating a time-series change of an RR interval measured from the ECG waveform illustrated in FIG. 3;

FIG. 5 is a graph illustrating a time-series change of a prescribed frequency component in the RR interval illustrated in FIG. 4;

FIG. 6 is a graph illustrating a part of the graph illustrating a time-series change of an RRI variability component illustrated in FIG. 5, in which a vertical axis is enlarged;

FIG. 7 is a graph illustrating a time-series change of a degree of variation of the RRI variability component, which is calculated based on the RRI variability component illustrated in FIG. 6;

FIG. 8 is a graph illustrating an index value R calculated by performing a normalization processing on a SDNN illustrated in FIG. 7; and

FIG. 9 is a flowchart illustrating a procedure of an operation in a case where a processing apparatus 2 according to an aspect of the presently disclosed subject matter performs pain evaluation of a subject.

### DESCRIPTION OF EMBODIMENTS

### Configuration of Vital Signs Information Processing Apparatus

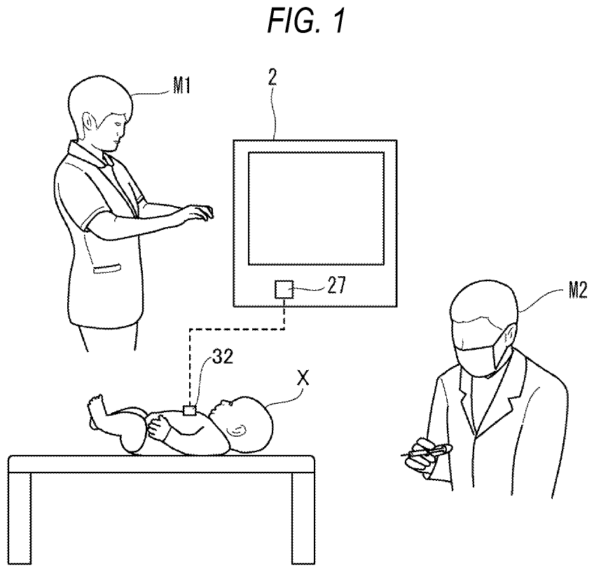

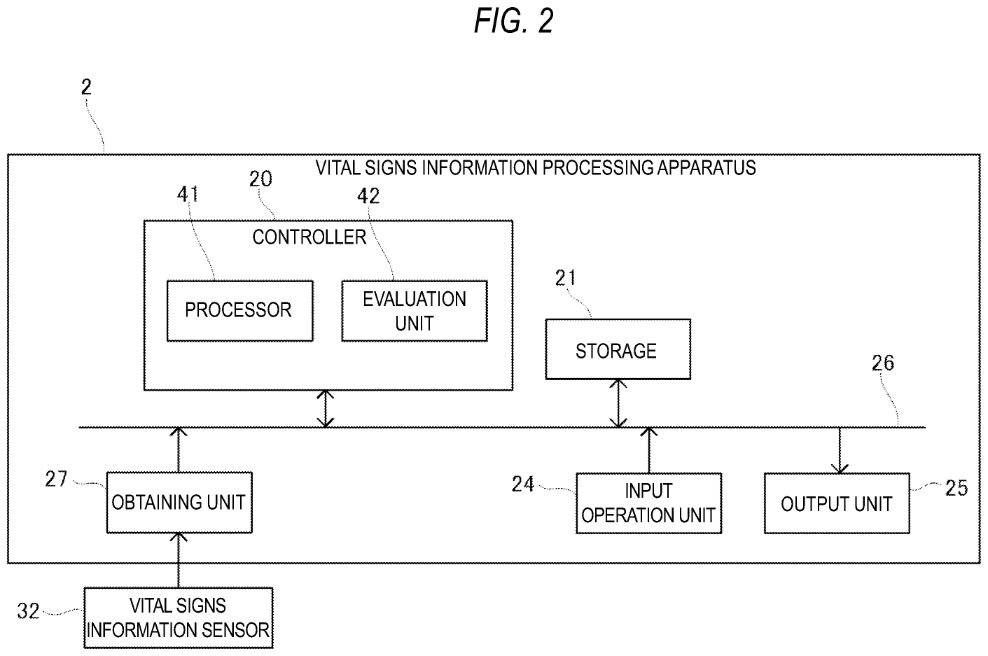

An exemplary embodiment of a vital signs information processing apparatus, a vital signs information processing method, and a non-transitory computer readable storage medium storing a vital signs information processing program according to the presently disclosed subject matter will be described with reference to the accompanying drawings. Hereinafter, a vital signs information processing apparatus 2 according to the present embodiment is referred to as the “processing apparatus 2”. FIG. 1 illustrates an example of use of the processing apparatus 2 according to the present embodiment. FIG. 2 illustrates a configuration of the processing apparatus 2 illustrated in FIG. 1.

Referring to FIG. 1, the processing apparatus 2 is an apparatus for evaluating pain of a subject X in a case where a treatment event such as blood collection is performed on the subject X. The treatment event is not limited to an event that causes pain to the subject X, and may be an event that may cause pain to the subject X. For example, the treatment event that causes pain is not limited to blood collection, and may be an event of drug administration, an examination involving punctures such as bone marrow examination, or an event in which a procedure involving incision or the like is performed. Medical workers M1 and M2 can understand whether the subject X feels pain by checking an evaluation result by the processing apparatus 2.

In FIG. 1, a newborn is illustrated as the subject X. Since it is difficult for a newborn to express the degree of pain by himself/herself through communication, it is particularly necessary for the medical workers M1 and M2 to understand the degree of pain of the newborn who is the subject X through the processing apparatus 2. The subject X is not limited to a newborn, and may be a child or an adult.

A vital signs information sensor 32 configured to obtain vascular dynamic information indicating a circulation state of blood is attached to the subject X. The vital signs information sensor 32 is, for example, an ECG sensor configured to measure an ECG signal of the subject X, and configured to transmit ECG data indicating the ECG signal to the processing apparatus 2, as the vascular dynamic information.

Referring to FIG. 2, the processing apparatus 2 can include a controller 20, a storage 21, an input operation unit 24, an output unit 25, and an obtaining unit 27. The controller 20, the storage 21, the input operation unit 24, the output unit 25, and the obtaining unit 27 are communicably connected to each other via a bus 26.

The obtaining unit 27 is an interface for communicably connecting the vital signs information sensor 32 to the processing apparatus 2. The obtaining unit 27 is configured to receive the vascular dynamic information transmitted from the vital signs information sensor 32, and configured to output the vascular dynamic information to the controller 20.

The controller 20 can include a processor 41 and an evaluation unit 42. The processor 41 is configured to perform a normalization processing on the vascular dynamic information received from the obtaining unit 27 to obtain an index value R used for pain evaluation of the subject X. The evaluation unit 42 is configured to perform the pain evaluation of the subject X, based on the index value R obtained by the processor 41. Details of the obtaining of the index value R by the processor 41 and the pain evaluation by the evaluation unit 42 will be described later.

The storage 21 is, for example, one or more flash memories, and configured to store programs and various data. The storage 21 may store a vital signs information processing program used for the pain evaluation by the controller 20. The storage 21 may store the vascular dynamic information on the subject X.

The input operation unit 24 is configured to receive input operations of the medical workers M1 and M2. The input operation unit 24 is, for example, a touch panel, a mouse, or a keyboard disposed on a monitor that is not illustrated. For example, after activating the processing apparatus 2, the medical worker M1 performs an input operation on the processing apparatus 2 immediately before performing a treatment event. The input operation unit 24 is configured to specify, as start time tx of a treatment event, time at which the input operation is performed or time after prescribed time elapses from the time at which the input operation is performed, and is configured to output start time information indicating the start time tx to the controller 20 via the bus 26.

### Obtaining of Index Value by Processor (Preliminary Processing)

Here, a procedure in which the processor 41 obtains the index value R of the pain evaluation based on the ECG data, which is an example of the vascular dynamic information, will be described.

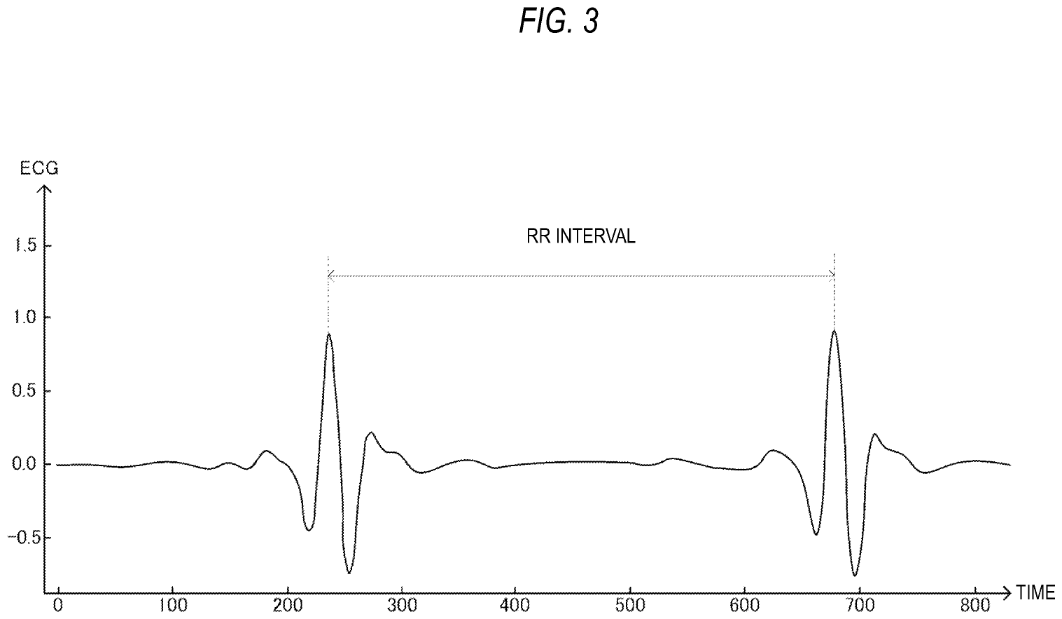

FIG. 3 is a graph illustrating an ECG waveform indicated by the ECG data output from the obtaining unit 27 illustrated in FIG. 2. Referring to FIG. 3, upon receiving the ECG data from the obtaining unit 27, the processor 41 detects an R wave generation time (hereinafter, referred to as “R timing”) of each heart rate of the subject X, based on the ECG waveform indicated by the ECG data. Then, the processor 41 measures an RR interval that is an interval between adjacent R timings.

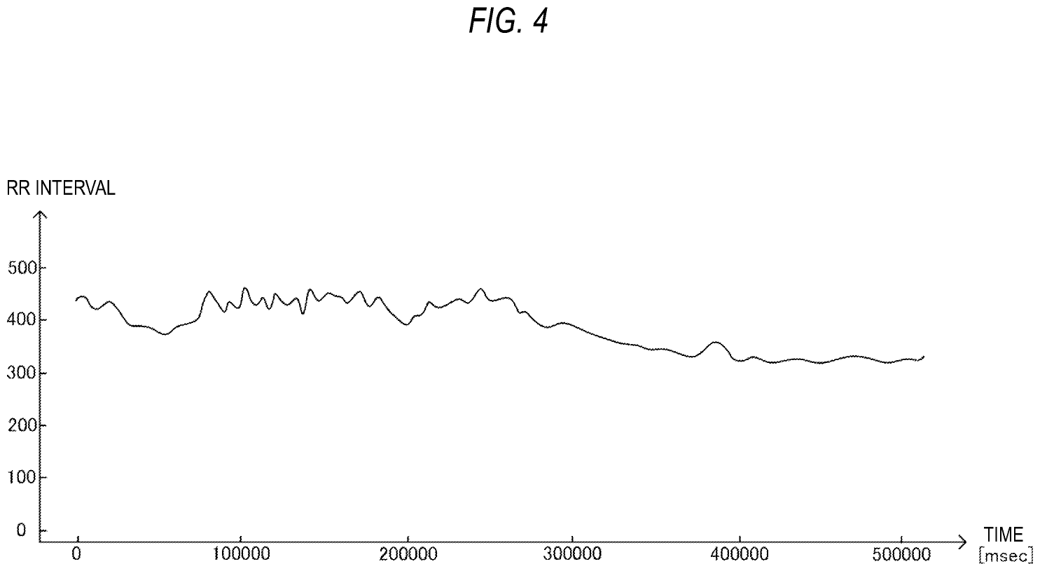

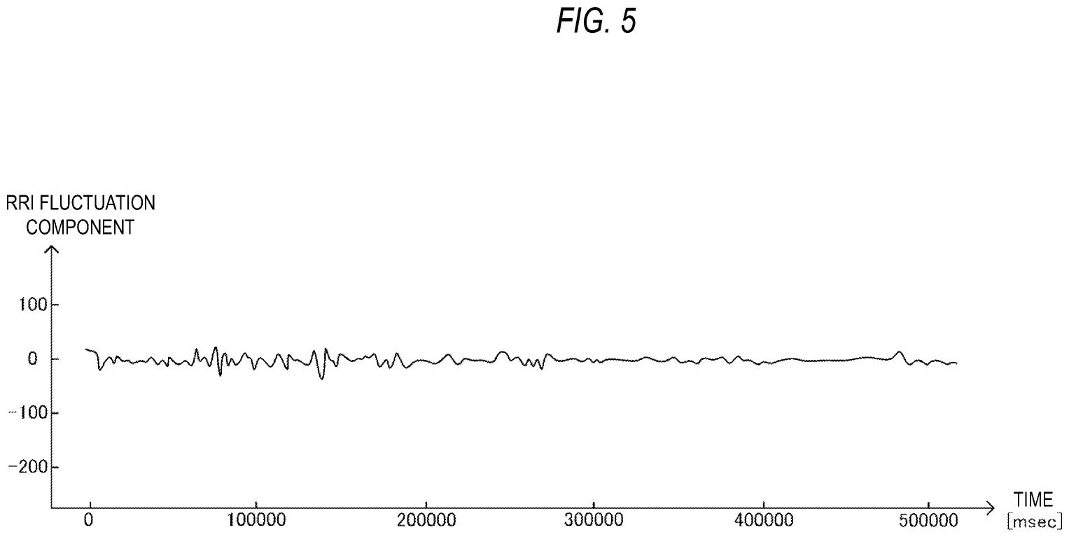

FIG. 4 is a graph illustrating a time-series change of the RR interval measured from the ECG waveform illustrated in FIG. 3. In FIG. 4, a horizontal axis represents time, and a vertical axis represents a value of the RR interval. FIG. 5 is a graph illustrating a time-series change of a prescribed frequency component (hereinafter referred to as “RRI variability component”) in the RR interval illustrated in FIG. 4. In FIG. 5, the horizontal axis represents time, and the vertical axis represents a value of the RRI variability component.

As illustrated in FIGS. 4 and 5, the processor 41 is configured to extract the RRI variability component from the RR interval measured based on the ECG waveform. Specifically, the processor 41 is configured to extract, as the RRI variability component, a frequency component included in a range from 0.2 Hz to 1.0 Hz from the RR interval.

Here, it is known that the frequency component included in the range of 0.2 Hz to 1.0 Hz in the RR interval reflects activities of parasympathetic nerves of the subject X. Specifically, an amplitude of the graph of the RRI variability component is large in a state in which the subject X does not feel pain and the parasympathetic nerves are active. On the other hand, the amplitude of the graph of the RRI variability component is small, in a state in which the subject X feels pain and the parasympathetic nerves are not active.

That is, pain of the subject X can be evaluated by quantifying the magnitude of the amplitude of the graph of the RRI variability component. For this reason, the processor 41 is configured to quantify the magnitude of the amplitude of the graph of the RRI variability component by calculating the degree of variation of the RRI variability component illustrated in FIG. 5. The degree of variation is standard deviation of normal to normal interval (SDNN), variance, entropy, or the like.

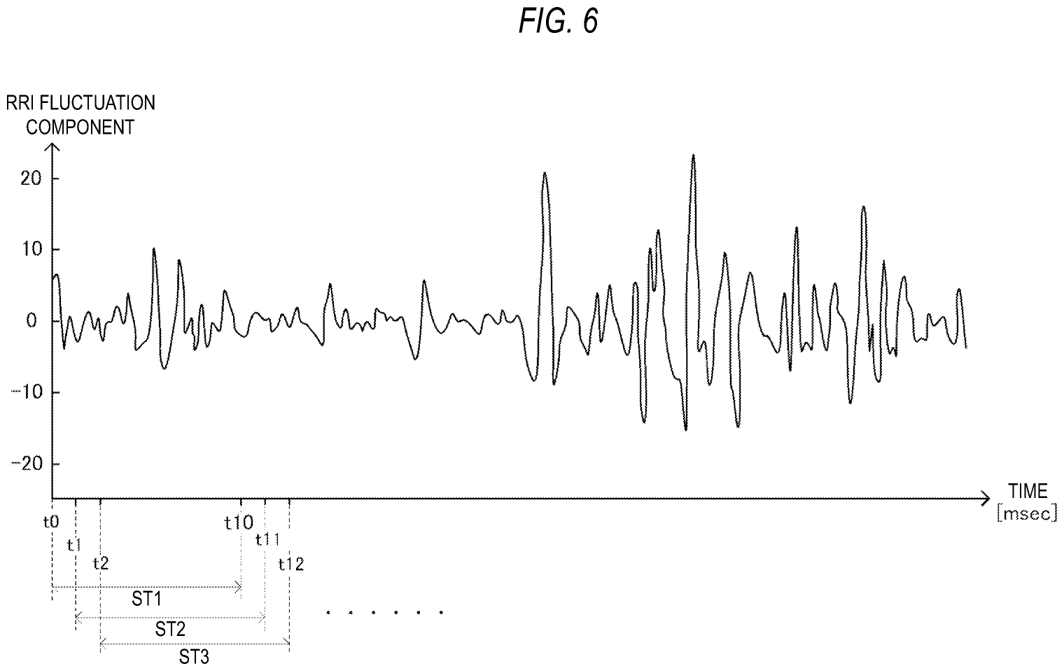

FIG. 6 is a part of the graph illustrating the time-series change of the RRI variability component illustrated in FIG. 5, in which the vertical axis is enlarged. Referring to FIG. 6, more specifically, the processor 41 is configured to set a plurality of sub-calculation periods ST for a period including the start time tx of the treatment event, and is configured to calculate the degree of variation of the RRI variability component for each sub-calculation period ST. The length of the sub-calculation period ST is, for example, 10 seconds. Consecutive sub-calculation periods ST include periods that overlap each other.

Specifically, for example, the processor 41 is configured to set an activation timing of the processing apparatus 2 as time t0, and configured to set a period from the time t0 to time t10 after 10 seconds as a sub-calculation period ST1. The processor 41 is configured to set, as a sub-calculation period ST2, a period from time t1 one second after the time t0 to time t11 after 10 seconds. The processor 41 is configured to set, as a sub-calculation period ST3, a period from time t2 one second after the time t1 to time t12 after 10 seconds. In this manner, the processor 41 is configured to calculate the degree of variation of the RRI variability component for each set sub-calculation period ST.

The consecutive sub-calculation periods ST may not include periods that overlap each other. The length of the sub-calculation period ST is not limited to 10 seconds.

### Obtaining of Index Value by Normalization Processing

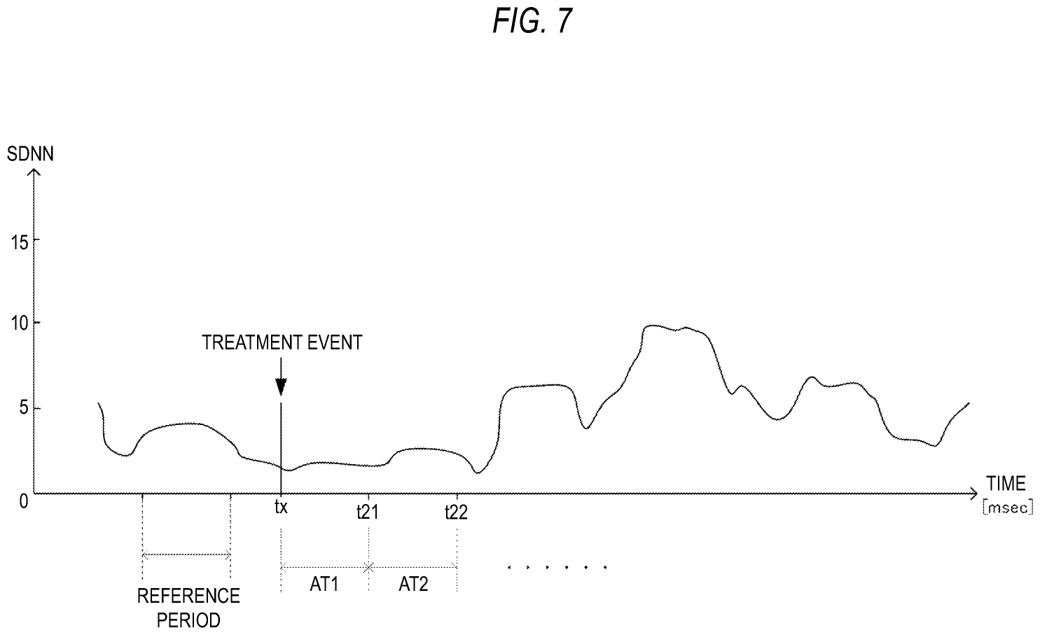

FIG. 7 is a graph illustrating a time-series change of the degree of variation of the RRI variability component, which is calculated based on the RRI variability component illustrated in FIG. 6. The graph illustrated in FIG. 7 illustrates a time-series change of the SDNN of the RRI variability component as an example of the degree of variation of the RRI variability component.

Referring to FIG. 7, the processor 41 is configured to perform the normalization processing on the calculated SDNN. That is, the processor 41 is configured to calculate, as the normalization processing, a ratio of the SDNN in a period after the start time tx of the treatment event to the SDNN in a period before the start time tx. Accordingly, the processor 41 is configured to obtain the index value R used for the pain evaluation.

### (i) Calculation of Reference Value BS

More specifically, for example, upon receiving start time information from the input operation unit 24, the processor 41 is configured to set a part of the period before the start time tx indicated by the start time information, as a reference period. The length of the reference period is, for example, 15 seconds. In the example illustrated in FIG. 7, 15 seconds in the period before the start time tx is set as the reference period.

Then, the processor 41 is configured to calculate the reference value BS used for the pain evaluation of the subject X using the SDNN, based on the vascular dynamic information obtained in the reference period. Specifically, the processor 41 is configured to calculate, as the reference value BS, an average value or a median value of the SDNN in the reference period.

### (ii) Calculation of Target Value V

The processor 41 is configured to calculate the target value V used for the pain evaluation of the subject X using the SDNN, based on the vascular dynamic information obtained after the start time tx. More specifically, the processor 41 is configured to divide the period after the start time tx into a plurality of target value calculation periods AT. The length of the target value calculation period AT is, for example, 15 seconds.

Specifically, the processor 41 is configured to set time t21 15 seconds after the start time tx, as a target value calculation period AT1. The processor 41 is configured to set time t22 15 seconds after the time t21, as a target value calculation period AT2. The processor 41 is configured to calculate, as the target value V, the average value or the median value of the SDNN, for each target value calculation period AT set as described above.

Consecutive target value calculation periods AT may include periods that overlap each other, as in the sub-calculation periods ST illustrated in FIG. 6. The length of the target value calculation period AT is not limited to 15 seconds.

(iii) Calculation of Index Value R

The processor 41 is configured to perform the normalization processing for each target value calculation period AT, and configured to calculate a ratio of the target value V to the reference value BS, as the index value R. Specifically, the processor 41 is configured to calculate a value (R1=V1/BS) obtained by dividing the target value V1 by the reference value BS, as an index value R1 in the target value calculation period AT1. The processor 41 is configured to calculate a value (R2=V2/BS) obtained by dividing the target value V2 by the reference value BS, as an index value R2 in the target value calculation period AT2. The processor 41 is configured to output the calculated index values R to the evaluation unit 42 illustrated in FIG. 2.

In the example described above, the processor 41 is configured to obtain the SDNN of the RRI variability component by performing the preliminary processing on the ECG data, which is an example of the vascular dynamic information, and is configured to calculate the index value R by performing the normalization processing on the obtained SDNN. However, the processor 41 is not limited to performing the preliminary processing, and may be configured to perform the normalization processing on a value indicated by the vascular dynamic information without performing the preliminary processing, for example.

### Pain Evaluation by Evaluation Unit

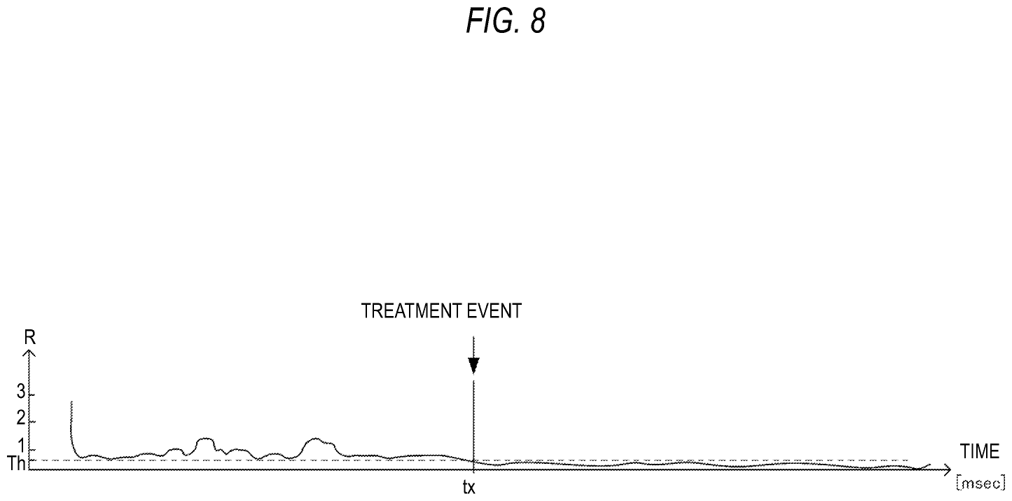

FIG. 8 is a graph illustrating the index value R calculated by performing the normalization processing on the SDNN illustrated in FIG. 7. In FIG. 8, the horizontal axis represents time, and the vertical axis represents the index value R. FIG. 8 illustrates a graph obtained by plotting the index value R every second as an example.

Referring to FIGS. 2 and 8, the evaluation unit 42 is configured to perform the pain evaluation of the subject X using the index value R output from the processor 41. For example, each time the evaluation unit 42 receives a newly calculated index value R from the processor 41, the evaluation unit 42 determines whether the index value R is equal to or less than a threshold Th. Specifically, the threshold Th is a value smaller than 1. In the example illustrated in FIG. 8, the index value R is equal to or less than the threshold Th after the start time tx of the treatment event. Thus, in a case where the index value R is equal to or less than the threshold Th, the evaluation unit 42 transmits an instruction signal to the output unit 25.

Upon receiving the instruction signal from the evaluation unit 42, the output unit 25 performs a warning processing for notifying the medical workers M1 and M2 that the subject X feels pain. For example, the output unit 25 is configured to perform display control such that a monitor or the like (not illustrated) displays a message indicating that the subject X feels pain. Accordingly, the medical workers M1 and M2 can understand that the subject X feels pain. The output unit 25 may be configured to perform output control such that, for example, a warning sound is output as the warning processing in addition to the display control.

The evaluation unit 42 may be configured to perform the pain evaluation according to the PIPP or PIPP-R described above. In this case, for example, the evaluation unit 42 can use the index value R calculated by the processor 41, as one of indexes set in the PIPP or the PIPP-R. The evaluation unit 42 may be configured to perform final pain evaluation by comprehensively determining an evaluation result of the pain evaluation, based on the index value R and an evaluation result of the pain evaluation according to the PIPP or PIPP-R.

The processor 41 and the evaluation unit 42 may be provided in different devices. For example, the processing apparatus 2 may not include the evaluation unit 42 or the output unit 25 illustrated in FIG. 2, but transmit the index value R calculated by the processor 41 to another device including the evaluation unit 42 and the output unit 25.

### Procedure of Operation

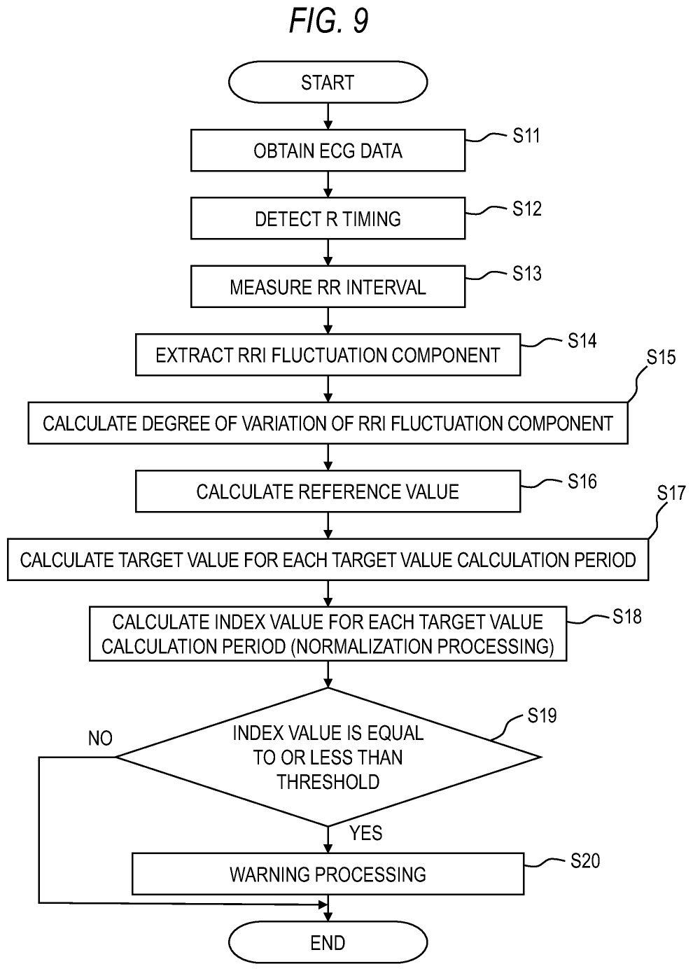

FIG. 9 is a flowchart illustrating a procedure of an operation in which the processing apparatus 2 according to an aspect of the presently disclosed subject matter performs the pain evaluation of the subject X using the vital signs information processing method. Here, the procedure of the operation in which the processing apparatus 2 performs the pain evaluation based on the ECG data will be described.

Referring to FIG. 9, in a case where an activation operation is performed by, for example, the medical worker M1, the processing apparatus 2 first obtains the ECG data transmitted from an ECG sensor that is an example of the vital signs information sensor 32 (step S11).

Next, the processing apparatus 2 detects the R timing of each heart rate, based on the ECG waveform indicated by the obtained ECG data (step S12), and measures the RR interval that is the interval between adjacent R timings (step S13). The processing apparatus 2 may perform a linear interpolation processing on a plurality of measured RR intervals.

Next, the processing apparatus 2 extracts a prescribed frequency component from the measured RR interval. Specifically, the processing apparatus 2 extracts a frequency component included in the range from 0.2 Hz to 1.0 Hz in the RR interval as the RRI variability component (step S14).

Next, the processing apparatus 2 sets a plurality of sub-calculation periods ST for the period including before and after the start time tx of the treatment event for the subject X, and calculates the degree of variation of the RRI variability component for each sub-calculation period ST (step S15).

Next, the processing apparatus 2 sets, as the reference period, a part of the period before the start time tx of the treatment event, and calculates, as the reference value BS, an average value or a median value of the degree of variation of the RRI variability component in the reference period (step S16).

Next, the processing apparatus 2 divides the period after the start time tx of the treatment event into a plurality of target value calculation periods AT, and calculates, as the target value V, an average value or a median value of the degree of variation of the RRI variability component, for each target value calculation period AT (step S17).

Next, the processing apparatus 2 performs the normalization processing for each target value calculation period AT, and calculates a ratio of the target value V to the reference value BS, as the index value R (step S18). Next, the processing apparatus 2 determines whether the calculated index value R is equal to or less than the threshold Th (step S19).

In a case where it is determined that the index value R is equal to or less than the threshold Th (“YES” in step S19), the processing apparatus 2 performs the warning process for notifying the medical workers M1 and M2 that the subject X feels pain (step S20). On the other hand, in a case where it is determined that the index value R is larger than the threshold Th (“NO” in step S19), the processing apparatus 2 does not perform the warning processing.

The vascular dynamic information is not limited to the ECG waveform of the subject X, and may be a pulse waveform of the subject X. In this case, the processor 41 of the processing apparatus 2 obtains, for example, the pulse waveform instead of the ECG data in step S11, and detects a peak of an ejection wave in the pulse waveform in step S12. In step S13, the processor 41 measures an interval between adjacent peaks as the RR interval.

### MODIFICATIONS

In addition to the index value R calculated by the processor 41, the evaluation unit 42 may perform the pain evaluation of the subject X, based on one or a plurality of types of vital signs information other than the vascular dynamic information. The vital signs information is, for example, information indicating arterial oxygen saturation, information indicating a heart rate, information indicating blood pressure, information indicating pulse wave propagation time, information indicating corrected QT time (QTc), information indicating body temperature, or information indicating a perfusion index.

In this case, one or a plurality of vital signs information sensors 32 such as a pulse sensor, a blood pressure sensor, and a body temperature sensor are further connected to the obtaining unit 27 of the processing apparatus 2 illustrated in FIG. 2 in addition to the ECG sensor. For example, the evaluation unit 42 performs prescribed weighting on the index value R and a value indicated by the vital signs information, and performs the pain evaluation of the subject X based on comprehensive evaluation.

The processor 41 may perform the normalization processing described above on a part or all of the vital signs information other than the vascular dynamic information. For example, the processor 41 may calculate an index value obtained by performing the normalization processing on the perfusion index among values indicated by the vital signs information received from the obtaining unit 27. In this case, the evaluation unit 42 performs the pain evaluation of the subject X using the index value calculated based on the perfusion index, the index value R calculated based on the ECG data, and the value indicated by the vital signs information other than the information indicating the perfusion index.

As described above, in the vital signs information processing apparatus 2 according to an aspect of the presently disclosed subject matter, the obtaining unit 27 is configured to obtain the vascular dynamic information indicating the circulation state of blood of the subject X. The processor 41 is configured to perform the normalization processing on the vascular dynamic information to obtain the index value R of the pain evaluation of the subject X. Further, the processor 41 is configured to calculate the reference value BS based on the vascular dynamic information obtained in the reference period before the start of the treatment event related to the treatment for the subject X, configured to calculate the target value V based on the vascular dynamic information obtained after the start of the treatment event, and configured to calculate the ratio of the target value V to the reference value BS as the index value R, as the normalization processing.

In this manner, the index value R of the pain evaluation is automatically calculated, and thus the burden on an evaluator who performs the pain evaluation, that is, the medical worker, can be reduced, and variations in the evaluation depending on evaluators can be reduced. In addition, since the normalization processing is performed on the vascular dynamic information on the subject X and the index value R is obtained, an individual difference of the subject X can be eliminated, and the pain evaluation can be performed more accurately.

As described above, the treatment event in which the vital signs information processing apparatus 2 according to the aspect of the presently disclosed subject matter is used is an event that causes pain to the subject X, and the event that causes pain can include at least one of an event of administering a drug to the subject X, an event of performing an examination on the subject X, or an event of performing a procedure on the subject X.

With such a configuration, the medical worker can understand whether the subject X feels pain in a plurality of types of treatment events.

As described above, the vascular dynamic information obtained by the vital signs information processing apparatus 2 according to the aspect of the presently disclosed subject matter is information indicating the ECG waveform or the pulse waveform of the subject X.

In this manner, the pain evaluation of the subject X can be performed more accurately by using information affected by an activity state of the parasympathetic nerves. An ECG sensor for obtaining the ECG waveform is often attached to the subject X during the treatment, and the pain evaluation can be performed using a sensor in the related art.

As described above, in the vital signs information processing apparatus 2 according to the aspect of the presently disclosed subject matter, the processor 41 is configured to calculate the degree of variation in a value indicated by the vascular dynamic information, based on the vascular dynamic information, configured to divide the period after the start of the treatment event into a plurality of target value calculation periods AT, and configured to calculate the target value V for each target value calculation period AT, based on the degree of variation in each target value calculation period AT.

With such a configuration, the activity state of the parasympathetic nerves can be quantified.

As described above, in the vital signs information processing apparatus 2 according to the aspect of the presently disclosed subject matter, each target value calculation period AT can include a plurality of sub-calculation periods ST. The processor 41 is configured to calculate the degree of variation for each sub-calculation period ST. The processor 41 is configured to calculate, for each target value calculation period AT, an average value or a median value of the degree of variation in each of the plurality of sub-calculation periods ST, as the target value V.

With such a configuration, even when the degree of variation temporarily changes due to an influence of noise or the like, the influence can be avoided and more accurate evaluation can be performed.

As described above, in the vital signs information processing apparatus 2 according to the aspect of the presently disclosed subject matter, the consecutive sub-calculation periods ST include periods overlapping each other.

With such a configuration, since a plurality of sub-calculation periods ST are included in each target value calculation period AT, a more reliable target value can be calculated using a plurality of variations.

As described above, in the vital signs information processing apparatus 2 according to the aspect of the presently disclosed subject matter, the evaluation unit 42 is configured to perform the pain evaluation of the subject X, based on the index value R obtained by the processor 41. The obtaining unit 27 is further configured to obtain, as the vital signs information, at least one of information indicating the arterial oxygen saturation of the subject X, information indicating the heart rate of the subject X, information indicating the blood pressure of the subject X, information indicating the pulse wave propagation time of the subject X, information indicating the QTc of the subject X, information indicating the body temperature of the subject X, or information indicating the perfusion index of the subject X. Then, the evaluation unit 42 is configured to perform the pain evaluation of the subject X, based on one or a plurality of types of the vital signs information in addition to the index value R.

As described above, since the pain evaluation is performed using a plurality of types of indices, the pain evaluation can be performed with higher accuracy.

Although the embodiment of the presently disclosed subject matter has been described above, the technical scope of the presently disclosed subject matter should not be construed as being limited to the description of the present embodiment. The present embodiment is merely an example, and it is understood by those skilled in the art that various modifications of the embodiment are possible within the scope of the disclosed subject matters described in the claims. The technical scope of the presently disclosed subject matter should be determined based on the scope of the disclosed subject matters described in the claims and equivalents thereof.

What is claimed is:

1. A vital signs information processing apparatus comprising:
an obtaining unit configured to obtain vascular dynamic information indicating a circulation state of blood of a subject; and
a processor configured to perform a normalization processing on the vascular dynamic information to obtain an index value of pain evaluation of the subject,
wherein the processor is configured to:
calculate a reference value, based on the vascular dynamic information obtained in a reference period before a start of a treatment event related to a treatment for the subject;
calculate a target value, based on the vascular dynamic information obtained after the start of the treatment event; and
calculate a ratio of the target value to the reference value as the index value, as the normalization processing.

2. The vital signs information processing apparatus according to claim 1,
wherein the treatment event is an event that causes pain to the subject, and
the event that causes pain to the subject includes at least one of an event of administering a drug to the subject, an event of performing an examination on the subject, or an event of performing a procedure on the subject.

3. The vital signs information processing apparatus according to claim 1,
wherein the vascular dynamic information is information indicating an ECG waveform or a pulse waveform of the subject.

4. The vital signs information processing apparatus according to claim 3,
wherein the processor is configured to:
calculate a degree of variation in a value indicated by the vascular dynamic information, based on the vascular dynamic information;
divide a period after the start of the treatment event into a plurality of target value calculation periods; and
calculate the target value for each of the target value calculation periods, based on the degree of variation in each of the target value calculation periods.

5. The vital signs information processing apparatus according to claim 4,
wherein each of the target value calculation periods includes a plurality of sub-calculation periods,
the processor is configured to:
calculate the degree of variation for each of the sub-calculation periods; and
calculate, as the target value, an average value or a median value of the degree of variation for each of the plurality of sub-calculation periods, for each of the target value calculation periods.

6. The vital signs information processing apparatus according to claim 5,
wherein the consecutive sub-calculation periods include periods overlapping each other.

7. The vital signs information processing apparatus according to claim 1, further comprising:
an evaluation unit configured to perform the pain evaluation of the subject, based on the index value obtained by the processor,
wherein the obtaining unit is further configured to obtain, as vital signs information, at least one of information indicating arterial blood oxygen saturation of the subject, information indicating a heart rate of the subject, information indicating blood pressure of the subject, information indicating pulse wave propagation time of the subject, information indicating corrected QT time (QTc) of the subject, information indicating body temperature of the subject, or information indicating a perfusion index of the subject, and
the evaluation unit is configured to perform the pain evaluation of the subject, based on one or a plurality of types of the vital signs information in addition to the index value.

8. A vital signs information processing method of a vital signs information processing apparatus, the vital signs information processing method comprising:
obtaining vascular dynamic information indicating a circulation state of blood of a subject; and
performing a normalization processing on the vascular dynamic information to obtain an index value of pain evaluation of the subject,
wherein in the performing:
a reference value is calculated based on the vascular dynamic information obtained in a reference period before a start of a treatment event related to a treatment for the subject;
a target value is calculated based on the vascular dynamic information obtained after the start of the treatment event; and
a ratio of the target value to the reference value is calculated as the index value, as the normalization processing.

9. A non-transitory computer readable storage medium storing a vital signs information processing program used in a vital signs information processing apparatus, the program comprising instructions, when executed by a computer, causing the computer in the vital signs information processing apparatus to:
obtaining vascular dynamic information indicating a circulation state of blood of a subject; and
perform a normalization processing on the vascular dynamic information to obtain an index value of pain evaluation of the subject,
wherein in the performing:
a reference value is calculated based on the vascular dynamic information obtained in a reference period before a start of a treatment event related to a treatment for the subject;
a target value is calculated based on the vascular dynamic information obtained after the start of the treatment event; and
a ratio of the target value to the reference value is calculated as the index value, as the normalization processing.
